(19) United States
(12) Patent Application Publication
(10) Pub. No.: US 2023/0172843 A1
(43) Pub. Date: Jun. 8, 2023

(54) METHOD OF PREPARING PH-SENSITIVE CONTROLLED-RELEASE EMULSION HYDROGEL

(71) Applicant: SEOUL NATIONAL UNIVERSITY R&DB FOUNDATION, Seoul (KR)

(72) Inventors: Pahn Shick CHANG, Seoul (KR); Hui Su KIM, Uijeongbu-si (KR); Hyun Jong YU, Yongin-si (KR); Eun Hye YANG, Incheon (KR)

(73) Assignee: SEOUL NATIONAL UNIVERSITY R&DB FOUNDATION, Seoul (KR)

(21) Appl. No.: 17/858,562

(22) Filed: Jul. 6, 2022

(30) Foreign Application Priority Data
Dec. 3, 2021 (KR) 10-2021-0172283

(51) Int. Cl. A61K 9/00; A61K 47/36; A61K 9/107; A23D 7/005; A23D 7/04; A23L 29/231; A23L 29/275

(52) U.S. Cl. CPC A61K 9/0053; A23D 7/04; A23D 7/0053; A23L 29/231; A23L 29/275; A61K 9/107; A61K 47/36; A23V 2002/00

(57) ABSTRACT

Disclosed is a method of preparing a pH-sensitive controlled-release emulsion hydrogel using chitosan and pectin. More particularly, disclosed is a method of preparing a pH-sensitive controlled-release emulsion hydrogel that is capable of entrapping various substances to be encapsulated and is stable in gastrointestinal conditions, but upon exposure to neutral pH after passing through the stomach, releases the entrapped emulsion in response to the pH.

### CROSS-REFERENCE TO RELATED APPLICATION

This application claims priority to Korean Patent Application No. 10-2021-0172283 filed Dec. 3, 2021, incorporated herein by reference in its entirety.

### BACKGROUND OF THE INVENTION

### Field of the Invention

The present invention relates to a method of preparing a pH-sensitive controlled-release emulsion hydrogel using chitosan and pectin and more particularly, to a method of preparing a pH-sensitive controlled-release emnlsion hydrogel that is capable of entrapping an emulsion and of releasing the emulsion in response to pH conditions.

### Description of the Related Art

Nano-encapsulation is widely used to protect active ingredients from the external environment and to increase bioavailability. In this regard, oil-in-water emulsions have been widely used as carriers of lipophilic physiologically active substances. However, emulsions become unstable when digested, thus affecting the bioavailability of physiologically active substances. Recently, research been conducted to improve the stability of emulsions in the gastrointestinal tract and to increase the bioavailability of physiologically active substances using a delivery system.

Recently, studies have been actively conducted on entrapment of the emulsion in the hydrogel to improve the stability of the emulsion. Such an encapsulation method has advantages of having high stability and usability compared to an emulsion surface-coating method. In order for this encapsulation method to be applicable to health functional foods or drug delivery systems, materials that are used must be safe for the human body and inorder to increase the bioavailability of physiologically active substances in the emulsion, research and development when on release control is required,

Meanwhile, a hydrogel refers to a polymer construct having a three-dimensional network structure containing an aqueous phase and is formed, by a covalent, or non-covalent bondbetween hydrophilic polymers. A hydrogel containing a variety of hydrophilic polymers has a high water and thus has excellent biocompatibility, so a great deal of research has been conducted to utilize the hydrogel as biomaterials. In particular, hydrogels sensitive to stimuli such as pH, temperature, electric field, magnetic field, light, and ultrasound waves can be used in a drug delivery system capable of conducting controlled release depending on the presence of stimuli.

### RELATED ART DOCUMENT

### Patent Document

(Patent Document 1) Korean Patent No. 10-1080428 (Oct. 31, 2011) discloses a method of preparing a pH-sensitive hydrogel using polyvinyl alcohol, a vinyl monomer, a crosslinking agent and a solvent.
(Patent Document 2) Korean Patent No. 10-1971834 (Apr. 17, 2019) discloses a pH-sensitive hydrogel containing a copolymer of carboxymethyl cellulose and hydroxyethyl acrylate.

### SUMMARY OF THE INVENTION

Therefore, the present invention has been made in view of the above problems, and it is one object of the present invention to provide a method of preparing a pH-sensitive controlled-release emulsion hydrogel that exhibits excellent release control in spite of using only materials safe to humans and thus is capable of increasing the bioavailability of a material encapsulated thereby.

It is another object of the present invention to provide a method of preparing a pH-sensitive controlled-release emulson hydrogel capable of entrapping a variety of materials to be encapsulated.

In accordance with the present invention, the above and other objects can be accomplished by the provision of a method of preparing a pH-sensitive controlled-release emulsion hydrogel, the method including (a) mixing an aqueous chitosan solution with an oil-in-water emulsion to prepare a chitosan-emulsion mixture, (b) heating the chitosan-emulsion mixture obtained in (a), and (c) adding a heated aqueous pectin solution to the heated chitosan-emulsion mixture obtained in (b) to prepare a chitosan-emulsion-pectin mixture and gelling the chitosan-emulsion-pectin mixture by cooling.

The aqueous chitosan solution is preferably an acidic aqueous solution.

The aqueous pectin solution is preferably an acidic aqueous solution.

The oil-in-water emulsion preferably contains a material encapsulated in an oil drop. The material is preferably a lipophilic physiologically active substance.

The pH-sensitive controlled-release emulsion hydrogel preferably releases the emulsion when the hydrogel is degraded in the small intestine.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other objects, features, and other advantages of the present invention will be more clearly understood from the followingdetailed description taken in conjunction with the accompanying drawings, in which:

FIG. 1 is a schematic diagram illustrating a method for preparing a pH-sensitive controlled-release emulsion hydrogel accordingto the present invention;

FIG. 2 shows the difference in gelation of the pH-sensitive controlled-release emulsion hydrogel according to the present invention depending on change in the chitosan concentration;

FIG. 3 shows the difference in gelation of the pH-sensitive controlled-release emulsion hydrogel according to the present invention depending on change in the pectin concentration;

FIG. 4 shows the result of SEM analysis of the pH-sensitive controlled-release emulsion hydrogel according to the present invention, illustrating structural change depending on the pectin concentration;

FIG. 5 shows the result of viscoelasticity analysis of the pH-sensitive controlled-release emulsion hydrogel according to the present invention, illustrating change in viscoelasticity depending on the pectin concentration; and

FIG. 6 shows the amount of emulsion that is released when the pH-sensitive controlled-release emulsion hydrogel according to the present invention is exposed to the gastrointestinal environment and the small intestine environment, illustrating change in the amount of emulsion released depending on the pectin concentration.

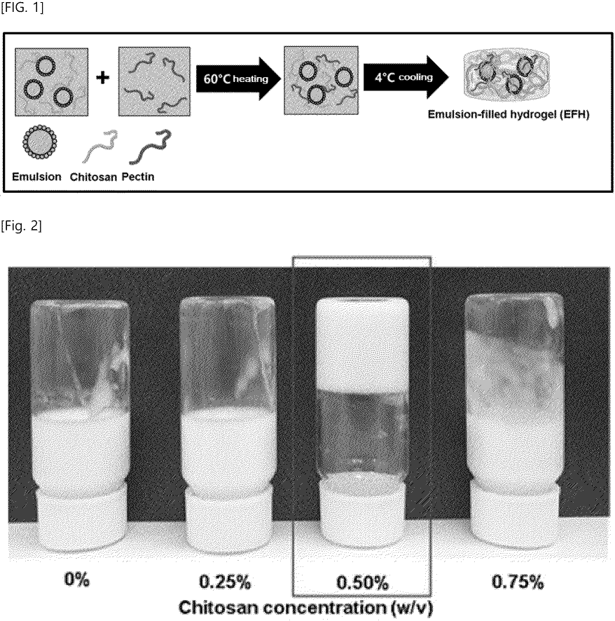

### DETAILED DESCRIPTION OF THE INVENTION

Hereinafter, the present invention will be described in detail with reference to the annexed drawings.

The present invention provides a method of preparing a pH-sensitive controlled-release emulsionhydrogel, including (a) mixing an aqueous chitosan solution with an oil-in-water emulsion to prepare a chitosan-emulsion mixture, (b) heating the chitosan-emulsicn mixture obtained in (a), and (c) adding a heated aqueous pectin solution to the heated chitosan-emulsionmixture obtained in (b) to prepare a chitosan-emulsion-pectin mixture and gelling the chitosan-emulsion-pectin mixture by cooling,

Conventionally known pH-sensitive controlled-release formulations exhibit poor at release control, for example, exhibit partial release even in low acidity conditions, or slow release in neutral conditions, Also, conventional pH-sensitive controlled-release formulations have a problem of being inapplicable to systems for increasing bioavailability through ingestion of drugs or functional food compositions due to the use of materials harmful to humans. Also, these formulations have a drawback of being inapplicable to various drugs or functional food compostions due to the limited type of substances that can be supported.

However, the present invention is capable of solving the above problems by incorporating an emulsion using chitosan and pectin as hydrogel skeletons and preparing an emulsion-containing hydrogel through cold-set gelation without using chemical substances in acidic conditions. The pH-sensitive controlled-release emulsion hydrogel according to the present invention is produced using materials that are safe for the human (chitosan and pectin) and is highly excellent in controlling release even after the emulsion is entrapped. In addition, the pH-sensitive controlled-release emulsion hydrogel may encapsulate a material in oil drops of the emulsion and is thus useful as an effective oral delivery system of the material.

Hereinafter, each step of the method of preparing a pH-sensitive controlled-release emulsion hydrogel according to the present invention will be described in detail.

### Step (a): Mixing Chitosan Aqueous Solution With Oil-In-Water Emulsion to Prepare Chitosan-Emulsion Mixture

This step is a process of mixing an aqueous chitosan solution with an oil-in-water emulsion.

The aqueous chitosan solution is preferably prepared by adding chitosan to an aqueous acidic solution. Chitosan may be prepared by deacetylation of chitin, which is a natural polysaccharide synthesized through β-(1->4)-linkages of N-acetyl-D-glucosamine, which can be obtained from cell walls of crustaceans such as crabs, common shrimp and crayfish, mollusks such as cuttlefish, insects, and bacteria. Also, chitosan, which is a polysaccharide obtained from natural products, has been reported to have excellent biocompatibility and biodegradability as well as physiological activities such as hemostasis, cholesterol removal, blood sugar control regulation, and liver function improvement, and is a cationic polymer that is positively charged at a pH of 6.5 or less.

Meanwhile, the oil-in-water emulsion preferably contains a material to be encapsulated in oildrops . For example, the oil-in-water emulsion may be prepared by dissolving lipophilic physiologically active material in an oil (oil drop) of an emulsion. Accordingly, the pH-sensitive controlled-release emulsion hydrogel according to the present invention can support a lipophilic physiologically active material.

### Step (b) : Heating Chitosan-Emulsion Mixture Prepared In Step (a)

This step is a heating process for preparing the pH-sensitive controlled-release emulsion hydrogel. In this step, the temperature of the chitosan-emulsion mixture is elevated to break the bonds in the polymer, so that the polymer (chitosan) can be mixed well with the emulsion and cold-set gelation can be performed well.

Meanwhile, the heating is preferably performed such that the temperature of the chitosan-emulsion mixture and the aqueous pectin solution reaches 55 to 65° C. When the heating is performed within the temperature range defined above, during subsequent cooling, gelation is facilitated. Also, the heating is perferably performed within the temperature range defined above because chitosan and pectin are not deformed or destroyed by heat. In addition, when heating is performed within the temperature range defined above, the physiologically active material supported in the oil drops is not deformed or destroyed by heat.

### Step (c): Adding Heated Aqueous Pectin Solution to Heated Chitosan-Emulsion Mixture, Followed by Cooling to Realize Gelation

This step is a process for completing the preparation of the pH-sensitive controlled-release emulsion hydrogel. In this step, the heated chitosan-emulsion mixture is added to the heated, aqueous pectin solution to prepare a chitosan-emulsion-pectin mixture, and then the chitosan-emulsion-pectin mixture is gelled through cooling.

Pectin is a polysaccharide that contains, as a main component, polygalacturonic acid formed by α-1,4 linkages of galacturonic acid, which can be obtained from citrus fruits such as oranges, tangerines, and apples. Pectin is a main component of cells of organisms, has excellent biocompatibility, and is an anionic polymer that is negatively charged in a wide pH range.

The aqueous pectin solution used herein has preferably been heated to 55 to 65° C. When the aqueous pectin solution is heated to the above temperature range, the bonds in the polymer are broken and the subsequent cold-set gelation is performed well.

In this step, the pH of the chitosan-emulsion-pectin mixture is preferably 1 to 3. If necessary, a pH adjusting agent may be added. The preparation method of the present invention is performed using chitosan and pectin, and is based on the characteristics that chitosan is a cationic polymer that is positively charged at a pH of 6.5 or less, whereas pectin is an anionic polymer that is negatively charged in a wide pH range. At a pH of 1 to 3, chitosan is positively charged to form an ionic bond with negatively charged pectin, thereby forming a pH-sensitive-release controlled emulsion hydrogel.

Meanwhile, in this step, the cooling is preferably carried out by allowing the mixture to stand in a refrigerator at 2 to 6° C. for 3.5 to 4.5 hours.

Meanwhile, in this step, the chitosan-emulsion-pectin mixture preferably has a concentration of 0.4-0.6% (w/v) of chitosan, and a concentration of 0.65-1.1% (w/v) of pectin. As can be seen from the following example, when a pH-sensitive controlled-release emulsion hydrogel was prepared at the predetermined concentration, it protected the well-encapsulated emulsion in an acidic environment (gastrointestinal environment), and released more than 95% of the emulsion in a neutral environment (small intestine environment) within 1 hour.

Hereinafter, the present invention will be described in more detail with reference to the following examples. The scope of the present invention is not limited to the examples, and encompasses modifications of the technical concept equivalent thereto.

### Example 1: Preparation of pH-Sensitive Controlled-Release Emulsion Hydrogel of Present Invention

In Example 1, a pH-sensitive controlled-release emulsion hydrogel was prepared as follows.

### Preparation of Chitosan-Emulsion Mixture

1) Chitosan was dissolved in a 0.1 M HCl aqueous solution at a concentration of 2.0% (w/v).

2) An emulsion in which oil is emulsified in water at a concentration of 10% (w-oil/v-water) (using sodium caseinate as an emulsifier) was mixed with the aqueous chitosan solution in equal amounts to prepare a chitosan-emulsion mixture.

### Preparation of Aqueous Pectin Solution

1.5% (w/v) pectin was dissolved in a 0.05 M HCl aqueous solution and then was heated to 60° C. to prepare an aqueous pectin solution.

### Mixing of Chitosan-Emulsion Mixture With Aqueous Pectin Solution

The chitosan-emulsion mixture prepared above was heated to 60° C. and then mixed with the pectin aqueous solution prepared above in equal amounts.

### The Chitosan-Pectin-Emulsion Mixture was Charged in a Square Mold, Cooled and Stored at 4° C. to Prepare a pH-Sensitive Controlled-Release Emulsion Hydrogel

The chitosan used herein was obtained from shrimp shells, the pectin used herein, was obtained from the peel of citrus fruits, the oil used herein was obtained from soybean, and sodium caseinate used herein was obtained from cow’s milk.

### Experimental Example 1: Observation of Changes in Characteristics of pH-Sensitive Controlled-Release Emulsion Hydrogel Depending on Chitosan Concentration

In Experimental Example 1, the changes in the characteristics of the pH-sensitive controlled-release emulsion hydrogel depending on the concentration of chitosan were observed.

The pH-sensitive controlled-release emulsion hydrogel was prepared in the same manner as in Example 1, except that the final concentration of pectin was fixed at 0.75% (w/v), and the final concentration of chitosan was changed to 0%, 0.25%, 0.50%, or 0.75% (w/v).

Meanwhile, a vial containing the pH-sensitive controlled-release emulsion hydrogel was stood upside down and whether or not gelation occurred well was observed (FIG. 2). As can be seen from FIG. 2, when the final concentration of chitosan was 0.5% (w/v), the hydrogel did not fall, which means that gelation occurred.

### Experimental Example 2: Observation of Changes in Characteristics of pH-Sensitive Controlled-Release Emulsion Hydrogel Depending on Pectin Concentration

### 1) Introduction

In Experimental Example 2, the changes in the characteristics of the pH-sensitive controlled-release emulsion hydrogel depedning on the concentration of pectin were observed.

The pH-sensitive controlled-release emulsion hydrogel was prepared in the same manner as in Example 1, except that the final concentration of chitosan was fixed at 0.5% (w/v), and the final concentration of pectin was changed to 0.50%, 0.75%, 1.00%, 1.25%, and 1.50% (w/v).

### 2) Confirmation of Gelation

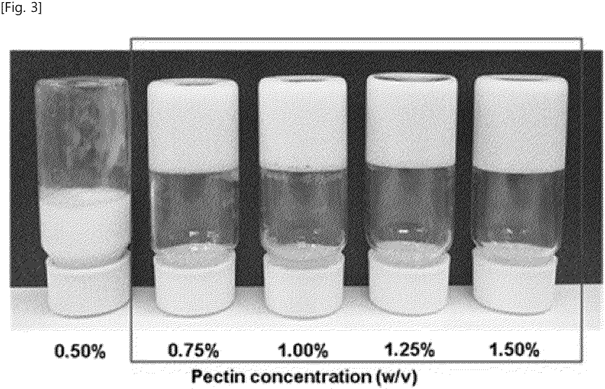

A vial containing the pH-sensitive controlled-release emulsion hydrogel was stood upside down and whether or not gelation occurred well was observed (FIG. 3). As can be seen from FIG. 3, when the concentration of pectin was 0.75% or more, gelation occurred.

### 3) Structural Analysis

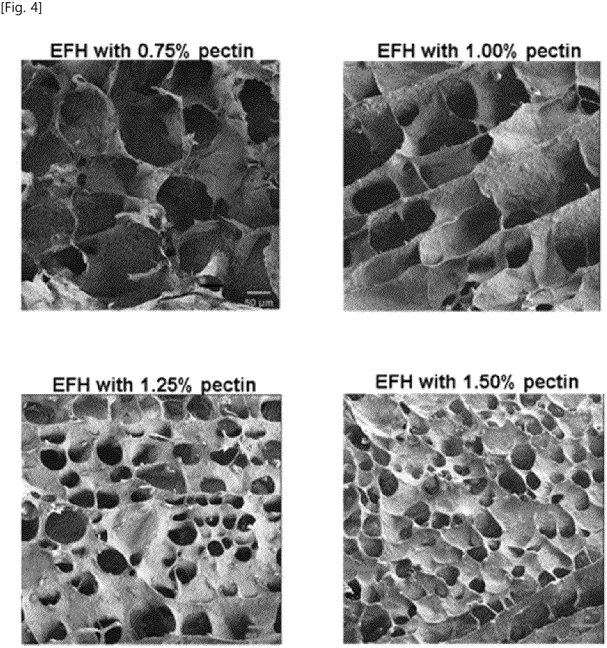

The microstructure of the prepared pH-sensitive controlled-release emulsion hydrogel (EFH) was observed by SEM analysis (FIG. 4). As can be seen from FIG. 4, as the concentration of pectin increased, the size of the structural space in the hydrogel decreased and thus a denser structure was formed.

### 4) Viscoelasticity Analysis

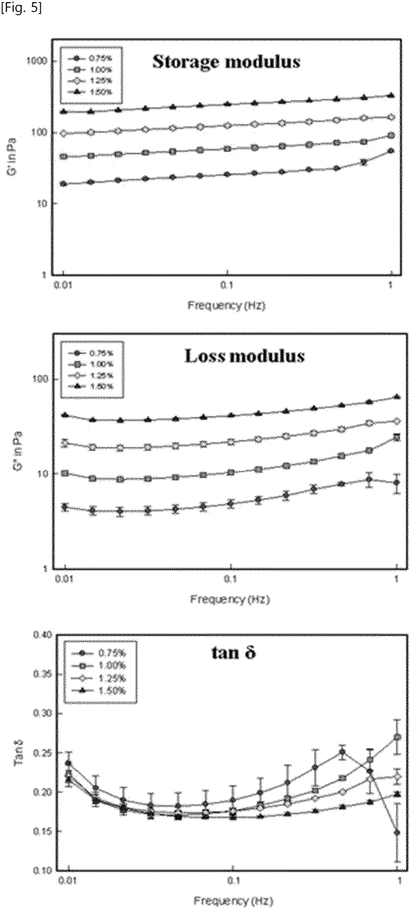

The viscoelasticity of the prepared pH-sensitive controlled-release emulsion hydrogel was analyzed through a frequency sweep test (FIG. 5). As can be seen from FIG. 5, as the concentration of pectin increased, the storage modulus increased, which supports that the structure became denser as the pectin concentration increased, like the result of SEM. Meanwhile, the gel collapsed at 1.00 Hz or higher.

### 5) Analysis of Physical Properties

The physical properties of the prepared pH-sensitive controlled-release emulsion hydrogel we analyzedTPA analysis (texture profile analysis) (Table 1).

TABLE 1

| Pectin concentration (%, w/v) | Hardness (N) | Resilience | Fracturability | Springiness (mm) |
|---|---|---|---|---|
| 0.75 | 0.47±0.02 ^{b} | 0.04±0.009^{b} | 0.07±0.03^{b} | 0.57±0.06^{d} |
| 1.00 | 0.43±0.06 ^{b} | 0.08±0.012^{a} | 0.08±0.02^{b} | 1.60±0.10^{c} |
| 1.25 | 0.42±0.04 ^{b} | 0.05±0.009^{a} ^{b} | 0.07±0.02^{b} | 1.45±0.06^{b} |
| 1.50 | 0.56±0.06 ^{a} | 0.07±0.016^{a} | 0.18±0.04^{e} | 2.70±0.13^{a} |

As can be seen from Table 1, the springiness significantly increased as the pectin concentration increased, which is consistent with the result of viscoelasticity analysis. In addition, fracturability, resilience, and hardness were poor, regardless of the pectin concentration.

### 6) Emulsion Release Test Depending on pH Change

In order to test at a pH similar to the actual intestine, a solution having the same pH of 2.0 as stomach and asolution having the same pH of 7.4 as the small intestine were prepared. In this experiment, curcumin was used as a material to be released and was encapsulated in oil phase of the emulsion.

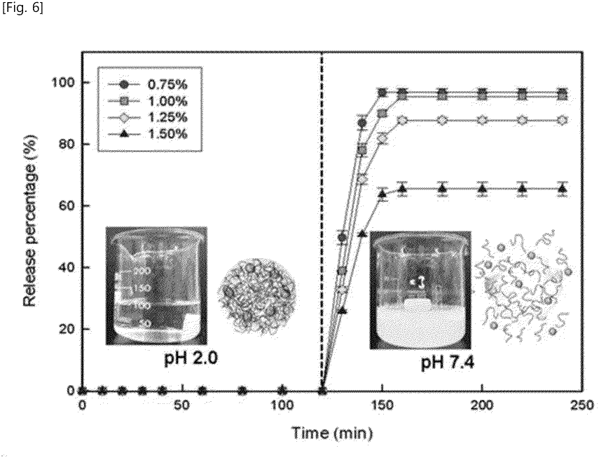

The prepared pH-sensitive controlled-release emulsion hydrogel was placed in the solution having a pH of 2.0, and after 2 hours, the solution was transferred to the solution having a pH of 7.4, and whether or not an emulsion was released was observed. At this time,the amount of emulsion that was released over time is shown in FIG. 6.

As can be seen from FIG. 6, at a pH of 2.0, the hydrogel was not decomposed whereas at a pH of 7.4, the hydrogel was decomposed and thus the emulsionwas released.In addition,it can be seen 1.50% (w/v), thefinal releaserate at pH 7.4 was decreased to 96.8% ,95.6% 87.8%, 65.5%,respectively. In addition, it seenthat,as the concentration of pectin increased, it can be seen that, as theconcentration of pectinincreased, the release rate decreased.

This means that the emulsion release rate and release amount can be controlled dependingon thepectin concentration hydrogel. Meanwhile,was considered that the of the hydrogel. Meanwhile, it was considered that the final release rate was not 100% because a part of the hydrogel was partially decomposed and maintained in the form of small segments.

As is apparent from the foregoing, the pH-sensitive controlled-release emulsion hydrogel prepared according to the method of the present invention is highly safe to humans, and protects the emulsion in an acidic environment, but releases the emulsion in a neutral environment, thus being useful as an effective oral delivery system for physiologically active substances in the emulsion.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A method of preparing a pH-sensitive controlled-release emulsion hydrogel, the method comprising:
(a) mixing an aqueous chitosan solution with an oil-in-water emulsion to prepare a chitosan-emulsion mixture;
(b) heating the chitosan-emulsion mixture obtained in (a); and
(c) adding a heated aqueous pectin solution to the heated chitosan-emulsion mixture obtained in (b) to prepare a chitosan-emulsion-pectin mixture and gelling the chitosan-emulsion-pectin mixture by cooling.

2. The method according to claim 1, wherein the aqueous chitosan solution is an acidic aqueous solution.

3. The method according to claim 1, wherein the aqueous pectin solution is an acidic aqueous solution.

4. The method according to claim 1, wherein the oil-in-water emulsion contains a material encapsulated in an oil drop.

5. The method according to claim 4, wherein the material is a lipophilic physiologically active substance.

6. The method according to claim 1, wherein the pH-sensitive controlled-release emulsion hydrogel releases the emulsion when the hydrogel is degraded in the small intestine.
